(19) United States
(12) Patent Application Publication
(10) Pub. No.: US 2009/0258438 A1
(43) Pub. Date: Oct. 15, 2009

(54) Method for universal biodetection of antigens and biomolecules

(76) Inventor: Wusi C. Maki, Coeur d'Alene, ID (US)

Correspondence Address: HOWARD EISENBERG, ESQ., 1220 LIMBERLOST LANE, GLADWYNE, PA, 19035, US

(21) Appl. No.: 12/383,389

(22) Filed: Mar. 24, 2009

Related U.S. Application Data
(60) Provisional application No. 61/123,703, filed on Apr. 9, 2008.

(51) Int. Cl. G01N 33/566; G01N 33/00

(52) U.S. Cl. 436/501; 436/94

(57) ABSTRACT

A universal signal molecule is generated in response to the presence within a biological fluid sample of a target agent. Two probes that bind to the target agent are provided within the sample and the target agent is captured, purified, and concentrated on a bead. One of the probes is attached to a signal nucleic acid that does not bind to the target agent. The signal nucleic acid is caused to be released from the probe, thereby generating a universal signal molecule. The presence of the universal signal molecule in the sample is detected, thereby providing for detection of the target agent within the sample.

This application claims priority from pending U.S. Provisional Patent Application No. 61/123,703, filed Apr. 9, 2008, which application is incorporated herein by reference in its entirety.

### FIELD OF THE INVENTION

The present invention pertains to the field of detection of antigens and biomolecules. In particular, the invention pertains to the field of detection of antigens and biomolecules by nano-biodetection methods, such as nanowire and nanotube methods.

### BACKGROUND OF THE INVENTION

The field-effect transistor (FET) is a type of transistor that relies on an electric field to control the conductivity of a channel in a semiconductor material. FETs have several terminals referred to as gate, drain, and source terminals and a body in which the gate, drain, and source terminals lie. The names of the terminals correspond to their functions. The gate blocks the passage of holes or free electrons or permits holes or free electrons to flow through by creating or eliminating a channel between the source and the drain. Current flows between the source terminal and the drain terminal if influenced by an applied voltage.

Nano-FET transistors can be used in the detection of antigens or biomolecules in a sample. In nano-FET transistors, there may not be a specific gate terminal. Instead, the nano-transistor contains a semiconductor device connected between source and drain with a specific biomolecular recognition element attached to the semiconductor. When the receptor binds to the semiconductor material, the charge on the substrate acts analogously to the voltage on the gate terminal, changing the current flowing between the drain and source terminals.

Examples of biomolecular receptors include antibodies and nucleic acids. A specific antibody linked to the gate point dielectric in a nano-FET is utilized to bind to a specific antigen of interest, such as a microbe or a polypeptide or protein. Specific nucleic acids, such as DNA or PNA (peptide nucleic acids) are used to bind nucleic acids, such as DNA or RNA of interest.

PNAs are nucleic acid mimics in which the sugar phosphate backbone has been replaced by a pseudo peptide-like backbone. Like DNA or RNA, a PNA will specifically and strongly bind to a DNA or RNA sequence of complementary sequence. Unlike DNA and RNA, however, PNA is electrically neutral, which provides an advantage in noise reduction when detecting biomolecules electronically. In addition, PNAs are resistant to degradation by nucleases.

In the detection of biomolecules or antigens, an agent of interest, such as a microbe like a bacterium or virus, a small molecule such as a drug, a polypeptide, a protein, or a nucleic acid, is captured by the biomolecular recognition element on the sensing surface of an FET chip. Microbes, polypeptides, and proteins may be bound by antigen-antibody interaction. Small molecules may be linked to a charge carrier molecule which is captured on the receptor surface by receptor-ligand interaction. DNA molecules may be captured by hybridization to specific DNA or PNA immobilized on the receptor surface.

Typically, target biomolecules are labeled, such as with biotin, and are captured on beads, such as strepavidin magnetic beads, in order to concentrate the target molecule from a complicated sample. Modified biotin, such as desthio-biotin, and/or modified strepavidin, such as nitro-strepavidin, may be used in order to facilitate the disassociation of the biotin/strepavidin complex. Excess amounts of D-biotin may be used to release the concentrated target molecule from the beads. The target molecule is then captured on the sensing surface. The binding of the biomolecule to the capture antibody, ligand, or nucleic acid is detected by a change in the electrical properties of the nano-FET.

A major problem in the field of nanowire biodetection is that, due to the high specificity of antigen-antibody interactions and nucleic acid hybridizations, a specific FET sensor utilizing a specific antibody or nucleic acid as a gate electrode recognition element must be produced for each antigen or biomolecule that is to be detected. This leads to tremendous cost and inefficiency in utilizing nano-FET detection technology and limits the number of antigens and biomolecules that are detected by nano-FET methods. Thus, a serious need exists for a universal detection method for antigens and biomolecules, which is universal in the sense that the nano-transistor structure and biomolecular surface are the same irrespective of the particular target antigen or biomolecule that is sought.

A method for providing a universal signal molecule for nano-biodetection was described as a bio-barcode assay in Goluch et al, Lab Chip, 6:1293-1299 (2006) for protein detection and in Stoeva et al, Angew. Chem. Int. Ed., 45:3303-3306 (2006) for DNA detection. The bio-barcode assay protocol is divided into two stages, a target separation stage in which a target molecule is recognized and a barcode DNA signal is produced and a barcode DNA detection stage, each of which occurs on separate areas of a microfluidic chip.

In the target separation stage, magnetic microparticles (MMP) that are functionalized with an antibody or nucleic acid that specifically binds or hybridizes to the protein or nucleic acid of interest are introduced into a micro-fluidic channel reactor on the separation area portion of the chip. A sample fluid is then flowed into the channel along with gold nanoparticle (NP) probes that are functionalized with an antibody or nucleic acid that specifically binds or hybridizes to the protein of interest. Thus, if the target of interest is present in the sample fluid, hybridized MMP-target-NP conjugate sandwiches are formed. The NP probes further contain strands of “barcode” DNA that does not bind to the target of interest. The MMP-target-NP conjugates are then immobilized to the channel wall with a magnet and the supernatant is washed away. Subsequently, heat denaturing or a reduction reaction in the presence of dithioreitol and vortexing is applied to the immobilized conjugates, which causes the dissociation of the barcode DNA from the NP probes.

In the barcode DNA detection stage, the released barcode DNA is transferred to a detection channel on the detection area portion of the chip, the bottom surface of which is functionalized with capture strands that are half-complementary to the barcode DNA. A second set of NP probes, functionalized with DNA that is complementary to the second half of the barcode DNA, is then introduced into the channel. Thus, the barcode DNA molecules permit the functionalized second set of NP probes to be hybridized to the surface of the channel. The presence of the functionalized second set of NP probes immobilized to the chip is detected and signifies the presence of the barcode DNA, which in turn signifies the presence of the target protein or DNA in the sample.

Several problems and shortcomings exist with the bio-barcode assay that are addressed and solved in the present invention as disclosed below. In the bio-barcode assay, gold nano-particles are used and it is difficult to control the amount of signal molecules that are attached to such particles. This can result in a variation of the detection, particularly in quantification detection. Additionally, oligonucleotide coated gold nano-particles are extremely “sticky” and bind to most test tubes and other materials non-specifically. Thus, the use of gold nano-particles presents problems of high noise background in the detection due to their non-specific attachment.

Additionally, the hybridizations that occur in the bio-barcode assay occur on solid surfaces. Such hybridization is less efficient than hybridization in a liquid. Additionally, the bio-barcode assay utilizes either de-ionized water at elevated temperatures or utilizes mechanical treatment in the presence of dithiothreitol to release signal molecules from the gold nano-particles. Such methods of release are difficult to control.

Accordingly, methods for generation of a universal signal for biodetection and for biodetection of a target agent that overcome the problems and shortcomings of the prior art are needed.

### BRIEF DESCRIPTION OF THE FIGURES

FIG. 1 is a diagrammatic representation of the method of the invention for generation of a universal signal (A to D) and of the method for biodetection of a nucleic acid target agent (A to E).

FIG. 2 is a diagrammatic representation of the method of the invention for generation of a universal signal ((A to D) and of the method for biodetection of a target agent other than a nucleic acid (A to E).

### DETAILED DESCRIPTION OF THE INVENTION

The present invention overcomes the problems and shortcomings of the prior art, specifically of the bio-barcode assay as, in the present invention, the ratio of the target agent and signal molecule that is detected is in a fixed ratio, typically 1:1, and the signal molecule has a defined molecular charge, which facilitates quantification detection. The present invention also does not utilize oligonucleotide coated gold nano-particles. Thus, the problem of “stickiness” associated with such nano-particles is avoided. In the present invention, hybridization reactions in the signal-generation phase occur within a sample, which is within a liquid, rather than on a solid gold nano-particle surface. Thus, hybridization in the present method is more efficient than in the bio-barcode assay. Additionally, the present invention uses an enzymatic process to release signal molecules, which is more efficient and controllable than the method of release in the bio-barcode assay.

In one embodiment, the invention is a method for generation of a universal signal molecule in response to the presence in a sample of an antigen or a biomolecule, which may be collectively referred to herein as a “target agent.” According to a preferred embodiment of the invention, a target agent in a sample is recognized, and this recognition event is translated into a universal signal molecule which is generated. The generated universal signal molecule carries a specific amount of electrical charge that is detectable by an electronic charge detecting sensor. Thus, the signal molecule may be used to indicate the presence of the target agent in the sample, such as by interacting with its specific chemical element on the surface of an electronic charge detecting sensor, such as a nano-FET, thereby eliciting a change in electrical properties of the FET, and indicating the presence of the target agent in the sample.

If desired, the signal molecule may be labeled or unlabeled, so as to be detectable by a label-free biodetection platform or by particular label biodetection platforms. As an example, the signal molecule may possess an optical element that is detectable by an optical biodetector. As another example, the signal molecule may possess a redox element that is detectable by electrochemical means.

Thus, according to a preferred embodiment of the invention, a target agent is bound to two probes, which may be nucleic acid or antibody probes. The first probe is also attached, directly or indirectly, to a magnetic bead. The second probe is attached, directly or indirectly, to a nucleic acid that does not bind to the target agent. The nucleic acid that does not bind to the target agent is then caused to be released from the second probe to become a universal signal molecule.

In another embodiment, the invention is a method for biodetection of a target agent. According to this embodiment of the invention, a target agent in a sample is recognized, this recognition event is translated into a universal signal molecule which is generated, and the generated signal molecule is recognized by biodetection platform, such as by a chemical element on the sensing surface of a nano-FET, by an optical detector, or by electrochemical means. The recognition by the chemical element on the nano-FET is indicated by a change in electrical properties of the FET, which change indicates the presence of the target agent in the sample.

In general, the recognition of the target agent in a sample involves capturing, purifying, and concentrating the target agent. The recognition utilizes a probe, such as a nucleic acid or an antibody that specifically binds to the target agent.

In cases in which the target agent is a DNA or RNA molecule, two nucleic acid probes are utilized. The first probe, referred to as a “capture probe,” is labeled, such as with biotin, and specifically hybridizes to the target agent. The second probe, referred to as a “signal probe,” contains two elements, a sequence that specifically hybridizes to the target agent and a target independent sequence. It is this target independent sequence portion of the signal probe that will become the universal signal molecule.

The two probes are exposed to a test sample. Target DNA or RNA molecules in the test sample hybridize to the two probes. Hybrids are captured, purified, and concentrated on magnetic beads by a biotin/strepavidin interaction. The signal molecule is then released from the hybrid through a specific nuclease digestion.

In cases in which the target agent is other than a nucleic acid two antibodies are used as probes for target recognition. An immobilized monoclonal antibody, such as on protein A/G magnetic beads, is exposed to the sample and is used to capture the target agent. The target molecules, such as proteins or other bio-agents, are captured and concentrated on the magnetic beads through an antibody-antigen interaction. A labeled, such as with biotin, second antibody is used to form a sandwich complex. A linker, such as nitro-strepavidin, is used as a linker between the second antibody and a labeled, such as with desthio-biotin, signal molecule, which is a single stranded nucleic acid. In the presence of competitor D-biotin, desthio-biotin labeled signal molecules, having a lower affinity for nitro-strepavidin, are released from the complex. The released oligonucleotide is the universal signal molecule.

In the method of detection of the invention, the recognition of the target agent, including capturing, purifying, and concentrating the target agent and generating the signal molecule, is referred to the “off-chip” process portion of the method because the recognition portion occurs independently of the FET. Following the off-chip portion, a subsequent “on-chip” portion involves the capturing of the signal molecule by the chemical element, typically a nucleic acid such as a PNA or an antibody, on the sensing surface of the nano-FET, which capturing affects the electronic properties of the nano-FET and generates a detectable electronic signal.

Because the capturing chemical element, typically a PNA, does not have to be complementary to any particular target agent, but rather is complementary to the signal molecule, the method of the invention provides universal detection of target agents such as microbes such as viruses and bacteria, polypeptides, proteins, small molecules, and nucleic acid sequences. The method of the invention thus permits a sensor having a nucleic acid capturing chemical element to be utilized for the detection of virtually any bio-agent without the need to change or modify the sensing surface of a nano-FET.

Any nano-FET device in which a nucleic acid, such as DNA or PNA is immobilized on its sensing surface, is suitable for the on-chip portion of the method of detection of the invention. Presently available nano-FET devices utilize a nanowire that is linear. Such linear nanowires are suitable for the method of the invention. In one preferred embodiment, a nanowire that is not a straight wire, such as a folded, wiggled, or spiral shaped nanowire is used for the nanowire in the nano-FET utilized in the on-chip portion of the method of detection.

The use of non-straight nanowires in an FET device, instead of a straight wire, may increase the electrical stability of the device and also may increase the sensing area of the nanowire. It has been shown that the I-V profiles of wiggled nano-FET devices show transistor behavior from −10.0 to 10.0V sweep back gate voltage with constant 0.5 bias voltage from drain to source.

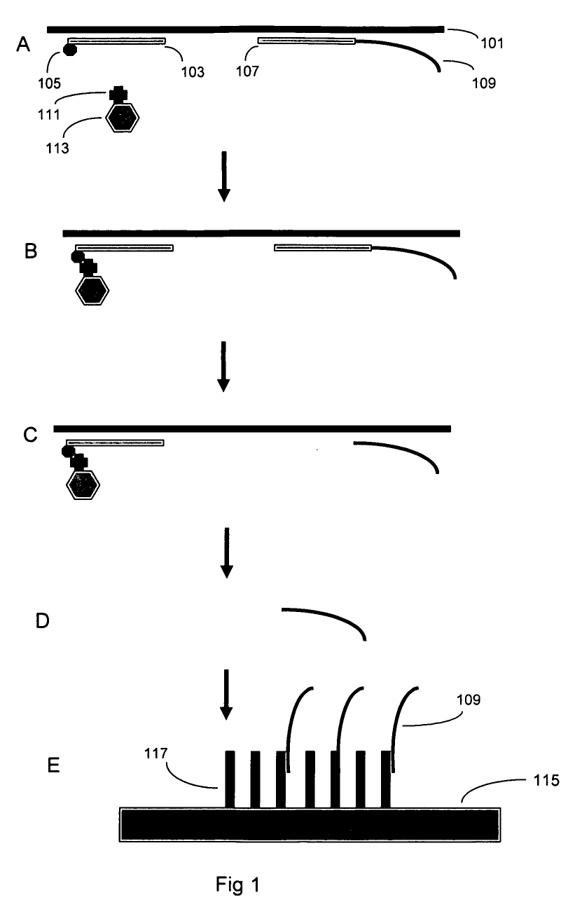

The method of the invention for generation of a universal signal from a nucleic acid target agent and the method of the invention for biodetection of a nucleic acid target agent are shown in FIG. 1. Sections A to D of FIG. 1 show the method for generation of a universal signal, which corresponds to the “off-chip” portion of the method for biodetection.

In section A, a capture probe 103 that has a sequence that is complementary to a first portion of a target nucleic acid agent 101 is labeled 105, which label may be biotin. A signal probe 107 has a target specific sequence that is complementary to a second portion of the target agent 101 and a tail portion 109 which will serve as the universal signal molecule. The capture probe 103 and the target specific portion of the signal probe 107 hybridize to the target agent 101.

In section B, the biotin label 105 interacts with strepavidin 111 that is coupled with magnetic beads 113. This provides a concentration and isolation of the target agent 101 hybridized to the two probes 103 and 107.

In section C, the hybridized target bound to the magnetic beads is exposed to a nuclease, such as an exonuclease or an endonuclease, such as T7 exonuclease, which digests the double stranded hybridized portion of the signal probe 107, thereby releasing the unhybridized single stranded tail portion 109 of the signal probe. Section D shows the released tail portion which functions as a universal signal molecule.

Section E shows the “on-chip” portion of the method for biodetection. A nano-FET device 115 is provided that contains on its sensing surface a capturing chemical element 117 that is a nucleic acid, such as a PNA, having a sequence that is complementary to the sequence of the signal molecule 109. The signal molecule hybridizes to the capturing chemical element, which causes a change in the electronic properties of the nano-FET device and generates a detectable electronic signal.

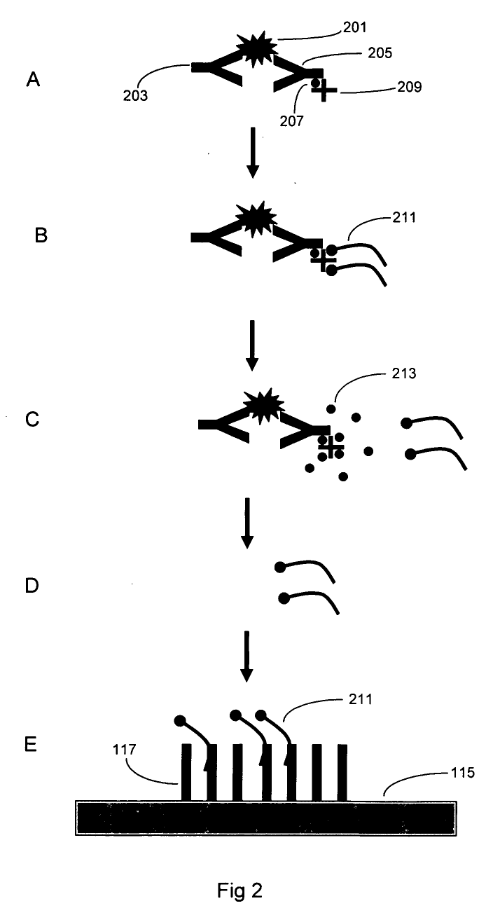

The method of the invention for generation of a universal signal from a target agent other than a nucleic acid and the method of the invention for biodetection of a target agent other than a nucleic acid, also referred to as immune-detection method, are shown in FIG. 2. Sections A to D of FIG. 2 show the method for generation of a universal signal, which corresponds to the “off-chip” portion of the method for biodetection.

In section A, a first antibody 203 immobilized on magnetic beads (not shown) is used to capture the target 201. A second antibody 205 that binds to the target is labeled, such as with biotin 207. Nitro-strepavidin 209 binds to the biotin label 207 on the second antibody.

In section B, an oligonucleotide 211 labeled with desthio-biotin binds is introduced and binds to the nitro-strepavidin 207. In section C, competitor D-biotin 213 displaces the desthio-biotin labeled oligonucleotide because the biotin 213 has higher affinity than does desthio-biotin label for the nitro-strepavidin. Section D shows the desthio-labeled oligonucleotide which functions as a universal signal molecule.

Section E shows the “on-chip” portion of the method for biodetection. A nano-FET device 115 is provided that contains on its sensing surface a capturing chemical element 117 that is a nucleic acid, such as a PNA, having a sequence that is complementary to the sequence of the oligonucleotide 211. The signal molecule hybridizes to the capturing chemical element, which causes a change in the electronic properties of the nano-FET device and generates a detectable electronic signal.

The methods of the invention may be used to generate a universal signal molecule in response to the presence in a sample of a nucleic acid, such as a DNA or RNA, or to determine the presence of a nucleic acid in a sample. The DNA molecule may be a methylated DNA, the detection of which may be useful in the diagnosis of cancer. The methods of the invention may be used to generate a universal signal molecule in response to the presence in a sample of a target agent other than a nucleic acid, for example a polypeptide or a protein, or to determine the presence of a target molecule other than a nucleic acid in a sample.

In another embodiment, the invention is a complex that includes a biomolecular target nucleic acid molecule, a labeled first nucleic acid probe hybridized to a first portion of the target nucleic acid, a second nucleic acid probe that contains a sequence that is hybridized to the target nucleic acid and a target independent sequence that is not hybridized to the target nucleic acid molecule. The complex of this embodiment of the invention is shown in FIG. 1, section A. Preferably, as shown in FIG. 1, section B, the label is a biotin label and this label is bound to strepavidin that is in turn coupled to a magnetic bead. Other ligand-receptor pairs may also be used for labeling the probe and capturing target on a magnetic bead.

In another embodiment, the invention is a complex that includes a biomolecular target other than a nucleic acid, such as a polypeptide or a protein, a first antibody bound to the target, a second antibody bound to the target, which second antibody is labeled with biotin, and nitro-strepavidin that is bound to the biotin. The complex of this embodiment of the invention is shown in FIG. 2, section A. Preferably, as shown in FIG. 2, section B, a oligonucleotide labeled with desthio-biotin is also bound to the nitro-strepavidin.

The method of the invention for generation of a universal signal molecule and the complexes of the invention may be used, as described above, in conjunction with a nano-FET device for biodetection of a target agent in a sample. The method for generation of a universal signal molecule and the complexes of the invention may also be used in conjunction with other applications and devices that provide biodetection of target agents. For example, the method for generation of a universal signal molecule or the complexes of the invention may be used with a cantilever nano-device, an electrochemical quartz crystal nano-balance, or an electrochemical impedance spectra biosensor. These other applications and devices provide label free detection of a target agent. The method for generation of a universal signal molecule or the complexes of the invention may also be used with a general electrochemical biosensor, such as by labeling the signal molecule with a redox element, or with a general optical detection method, such as by labeling the signal molecule with an optical element.

While preferred embodiments of the invention have been described in detail, it will be apparent to those skilled in the art that the disclosed embodiments may be modified. It is intended that such modifications be encompassed in the following claims. Therefore, the foregoing description is to be considered to be exemplary rather than limiting, and the scope of the invention is that defined by the following claims.

1. A method for generating a universal signal molecule in response to the presence in a sample of a target agent comprising providing within the sample a first probe that specifically binds to the target agent, providing within the sample a bead to which the first probe binds, providing within the sample a second probe that specifically binds to the target agent and that is attached to a signal nucleic acid that does not bind to the target agent, then causing the signal nucleic acid to be released from the second probe, thereby generating the universal signal molecule.

2. The method of claim 1 wherein the target agent is a nucleic acid.

3. The method of claim 2 wherein the first and second probes are nucleic acids.

4. The method of claim 3 wherein the first probe is labeled with a compound that binds to the bead.

5. The method of claim 4 wherein the label is biotin, the bead is coupled with strepavidin, and the label binds to the bead by a biotin/strepavidin interaction.

6. The method of claim 3 wherein the signal nucleic acid is released by action of a nuclease that digests double stranded nucleic acids.

7. The method of claim 1 wherein the target agent is other than a nucleic acid.

8. The method of claim 7 wherein the target agent is a polypeptide or a protein.

9. The method of claim 7 wherein the first and second probes are antibodies.

10. The method of claim 9 wherein the second probe and the signal nucleic acid are labeled and the label on the second probe and the label on the signal nucleic acid bind to the same molecule.

11. The method of claim 10 wherein the label is on the second probe binds to the molecule with higher affinity than does the label on the signal nucleic acid.

12. The method of claim 11 wherein the label on the second probe is biotin, the label on the signal nucleic acid is desthio-biotin, and the molecule is nitro-strepavidin.

13. The method of claim 12 wherein the signal nucleic acid is released from the nitro-strepavidin by competitor biotin.

14. The method of claim 1 wherein the signal molecule carries an electronic charge that is detectible by an electronic charge detecting sensor.

15. The method of claim 1 wherein the signal molecule possesses an optical element that is detectable by an optical detector.

16. The method of claim 1 wherein the signal molecule possesses a redox element that is detectable by electrochemical means.

17. The method of claim 1 wherein the signal molecule is detectable by a label free biodetection platform.

18. A method for biodetection of a target agent within a sample comprising capturing, purifying, and concentrating the target agent on beads within the sample, recognizing the target agent within the sample with a first and second probe, wherein the first probe binds to the target agent and to the beads, and wherein the second probe binds to the target agent and is attached to a signal nucleic acid that does not bind to the target molecule, then causing the signal nucleic acid to be released from the second probe, thereby generating a universal signal molecule, and causing the presence of the universal signal molecule to be detected on a detecting sensor, thereby biodetecting the target agent.

19. The method of claim 18 wherein the target agent is a nucleic acid.

20. The method of claim 18 wherein the target agent is not a nucleic acid.

21. The method of claim 20 wherein the target agent is a polypeptide.

22. The method of claim 18 wherein the universal signal molecule is not labeled with a label that is detectable by the detecting sensor.

23. The method of claim 18 wherein the universal signal molecule carries an electric charge and the detecting sensor is an electronic charge detecting sensor.

24. The method of claim 23 wherein the electronic charge detecting sensor is a nano-transistor.

25. The method of claim 18 wherein the universal signal molecule carries an optical element and the detecting sensor is an optical detector.

26. The method of claim 18 wherein the universal signal molecule carries a redox element and the detecting sensor is an electrochemical detector.

27. A complex comprising within a biologic fluid a biomolecular target nucleic acid molecule, a first nucleic acid probe hybridized to a first portion of the target nucleic acid molecule, a second nucleic acid probe hybridized to a second portion of the target nucleic acid molecule, and a target nucleic acid independent nucleic acid that is attached to the second nucleic acid probe.

28. A complex comprising within a biological fluid a biomolecular target other than a nucleic acid, a first antibody probe bound to the target, a second antibody bound to the target, a biotin label attached to the second antibody, and nitro-strepavidin bound to the biotin label.
